(19) United States
(12) Patent Application Publication
(10) Pub. No.: US 2003/0138850 A1
(43) Pub. Date: Jul. 24, 2003

(54) Selecting for antibody-antigen interactions in bacteria cells by employing a protein fragment complementation assay

(76) Inventors: Ekkehard Mossner, Zurich (CH); Holger Koch, Zurich (CH); Andreas Pluckthun, Zurich (CH)

Correspondence Address: HELLER EHRMAN WHITE & MCAULIFFE LLP, 1666 K STREET,NW, SUITE 300, WASHINGTON, DC, 20006, US

(21) Appl. No.: 10/273,358

(22) Filed: Oct. 18, 2002

Related U.S. Application Data
(60) Provisional application No. 60/329,763, filed on Oct. 18, 2001.

(51) Int. Cl. C12Q 1/68; G01N 33/53; G01N 33/554; G01N 33/569

(52) U.S. Cl. 435/7.1; 435/6; 435/7.32

(57) ABSTRACT

Methods are provided for the selection or screening of an antibody fragment library in bacterial cells by using a Protein Fragment Complementation Assay. The invention also provides nucleic acid molecules that encode fusion proteins, which contain a first part of a reporter molecule (e.g., a DHFR or beta-lactamase fragment), a linker, and an antibody or a functional antibody fragment. The foregoing readily can be adapted for recombinant expression, through the use of vectors and host cells, for example.

The present application claims the benefit, under 35 U.S.C. § 119(e), to U.S. provisional patent application Ser. No. 60/329,763, which is hereby incorporated by reference in its entirety.

### BACKGROUND OF THE INVENTION

1. Field of the invention

The invention provides methods for the selection and screening of an antibody fragment library in the cytoplasm of bacterial cells, e.g., E. coli, by using a Protein Fragment Complementation Assay. The invention also provides host cells comprising nucleic acid sequences encoding fusion proteins comprising fragments of a reporter protein, such as the bacterial enzyme DHFR, vectors comprising such fusion proteins and host cells harboring these vectors and nucleic acid sequences.

2. Background

The rapid rate of gene discovery by several large-scale genome projects has generated an immense amount of sequence data that are not matched by corresponding functional data. Monoclonal antibodies are a powerful tool for functional studies of newly discovered genes, yet the availability of such antibodies often is a limiting prerequisite for carrying out such studies. Several techniques are available that allow screening of large antibody libraries for the identification of specific binders against a target of interest. All of these methods have in common that the genetic information encoding the antibody of interest is physically linked to its phenotype, and the genes of selected antibodies can be reamplified and, thus, are readily available for further analysis. Suitable screening and selection techniques include, among others, phage display (Smith, 1985; Winter et al., 1994), ribosome display (Hanes et al., 1998) and surface display on yeast and bacteria (Boder & Wittrup, 1997; Georgiou et al., 1997).

In all these screening systems, the antibodies first are expressed and then, in a separate step, contacted with the purified antigen to screen for a specific antigen-antibody interaction. It would be advantageous, however, to simply coexpress the antibody and the antigen in the same environment, e.g. in the cytoplasm of a host cell.

But it has been generally understood that the folding of immunoglobulin domains requires the formation of conserved structural disulfide bonds. Therefore, as a general rule, antibodies cannot be functionally expressed in the reducing environment of the prokaryotic cellular cytoplasm (Wirtz & Steipe, 1999). The same disadvantage is thought to apply to the expression of other proteins if the formation of structural disulfide bonds is required for correct folding to yield functional protein.

Recently, a protein selection system has been developed that functions in E. coli, and that is based on the functional complementation of a reporter protein, e.g. dihydrofolate reductase (DHFR). This method is referred to as a Protein Fragment Complementation Assay (PCA). See Pelletier et al., 1998; Pelletier et al., 1999; WO 98/43120; WO 00/07038; U.S. Pat. No. 6,294,330 and U.S. Pat. No. 6,270,964, all of which are hereby incorporated by reference in their entirety. In a PCA, the reporter protein is dissected into two parts or fragments which, as such, cannot independently re-associate or interact with each other so as to regain the protein function that was lost by virtue of the cleavage. However, if two specifically interacting partners are respectively fused to the two parts or fragments of the cleaved reporter protein (i.e., one reporter fragment per specifically interacting partner), interaction of the fusion proteins results in the restoration of the reporter protein activity.

According to a particular PCA protocol, the gene of the murine DHFR (mDHFR), for example, can be dissected into two parts (fragment I and fragment II, see FIG. 1), and each part can be fused to a gene coding for a protein or peptide. The two plasmids encoding these constructs are co-expressed in E. coli. If the two proteins or peptides fused to the DHFR parts recognize and bind to each other, the two halves of mDHFR will come into close contact, and thereby restore mDHFR enzymatic activity.

E. coli DHFR, but not murine DHFR, is inhibited by the antibiotic trimethoprim (TMP). Thus, E. coli cells expressing functional mDHFR are able to grow on minimal medium in the presence of TMP. This system had been successfully applied to select for leucine-zipper domains with optimized binding properties in both a bait vs. library and a library vs. library selection (Pelletier et al., 1999; Arndt et al., 2000). This method works rapidly and reliably due to a negligible background of false positives. Alternative PCA strategies based on similar structural criteria have been suggested, e.g. using fragments of GST, or constructs comprising bleomycin binding protein fragments (see WO 98/34120).

One important advantage of this system for antibody screening and selection would be that, unlike phage display, antigens would not have to be produced and purified separately, because only the DNA of the antigen need be cloned to provide the mDHFR-fusions in situ. Accordingly, it would be highly desirable to develop a high-throughput method using a PCA system to screen antibody libraries against antigens or antigen libraries in the cytoplasm of E. coli without the need to prepare purified antigen or to isolate libraries of antibodies.

However, prior workers limited their investigation to leucine zipper interactions and did not attempt to develop a PCA method capable of selecting for antibody-antigen interactions. There could be multiple reasons for this. For one, the nature of an antibody-antigen interaction (e.g. protein size, tertiary and quarternary structure) is quite distinct from the leucine zipper interactions that previously were investigated. Accordingly, the regeneration of reporter protein function described by prior workers is not predictive of the probability of regenerating reporter protein function in the context of antibody-antigen interactions, regardless of the cellular environment (e.g., cytosol/cytoplasm or periplasm). In fact, prior to the present invention, there had been no reports of successful screening for specific antigen-antibody interactions using a reporter molecule expressed as two moieties (one reporter molecular moiety expressed as a fusion with the antigen, and the other moiety expressed as a fusion with the antibody or fragment thereof) where reporter molecule function was restored upon specific antigen-antibody interaction.

Furthermore, it had previously been understood that disulfide-containing molecules such as antibody fragments could not be successfully expressed in functional form in the cytoplasm, i.e., the environment where PCA was carried out. In particular, during the expression of antibodies in E. coli cytoplasm, the reducing environment hinders the formation of the intrachain disulfide bonds of the variable domain of the heavy and light chains (Cattaneo & Biocca, 1999). These disulfide bonds contribute about 4-5 kcal/mol to the overall stability of the scFv and, accordingly, antibodies are strongly destabilized in their reduced form (Frisch et al., 1996). Indeed, several scFv antibodies have been studied and shown to be sensitive to the absence of their stabilizing disulfide bonds (Glockshuber et al., 1992; Proba et al., 1995).

Several approaches have been tried to solve the problem of antibody expression in the cytoplasm. However, none has been shown to be generally applicable to the expression of antibody fragments in the bacterial cytoplasm.

For example, although it has been shown that some cysteine-free scFv fragments are stable enough that they can fold in the absence of the conserved disulfide bonds in both V_{H} and V_{L} chains and that they can be functionally expressed in E. coli cytoplasm (Chen et al., 1994; Proba et al., 1998; Jermutus et al., 2001), this result does not appear to be generally applicable to the great majority of scFv sequences. Moreover, it would be impractical or even impossible to eliminate disulfide containing antibodies from an antibody library of any significant size.

In another approach, several scFv antibodies have been tested for activity in a yeast two-hybrid assay, and a minority population showed apparent activity in the cytoplasm of the eukaryotic organism Saccharoinyces cerevisiae (Cattaneo & Biocca, 1999; Visintin et al., 1999; De Jaeger et al., 2000). A stability-engineered anti-GCN4 scFv was expressed in the cytoplasm of S. cerevisiae, and shown to be functional (Wörn et al., 2000). Once again, however, this result is not generally applicable to expression in prokaryotic systems.

In another example, several rounds of mutation and selection in vivo were used to derive an antibody fragment “with greatly improved expression in the bacterial cytoplasm” but of unknown function (Martineau et al., 1998). To this end, an antibody scFv fragment against β-galactosidase was isolated from a phage antibody library and random mutations were introduced in the scFv gene by PCR amplification to derive the scFv antibody fragment. Id. Martineau et al. reported that “[t]he reasons for high levels of [cytosolic] expression are not clear.” Id. It is apparent that this method is not generally applicable and, in particular, is unsuitable for use with antibody libraries containing a diverse range of binding activities. Accordingly, the expression and screening of antibody libraries in the cytoplasm has not been possible previously.

It is apparent, therefore, that new and improved methods for expressing and screening antibodies (e.g., parental antibody library clones), preferably in the bacterial cytoplasm, are greatly to be desired.

### SUMMARY OF THE INVENTION

Accordingly, it is an object of the present invention to provide methods to select or screen an antibody library in the cytoplasm of bacteria (cytosol) for binding to a binding target by using a reporter protein in a Protein Fragment Complementation Assay

It is also an object of the present invention to provide an improved method for the selection or screening of one or more members of an antibody fragment library for binding to a (poly)peptide/protein in the cytoplasm of bacterial host cells by using the DHFR Protein Complementation Assay.

It also is an object of the present invention to provide a method using bacterial host cells comprising nucleic acid sequences encoding fusion proteins comprising the parts of a reporter protein, such as a DHFR.

In accordance with this object of the present invention, there are provided nucleic acid sequences encoding a first fusion protein comprising a first part of a reporter protein, such as a DHFR or beta lactamase molecule, optionally a first linker, and an antibody fragment and a second fusion protein comprising the second part of the reporter protein (such as a DHFR molecule), optionally a second linker, and a (poly)peptide/protein.

In accordance with this object of the present invention, the activity of the reporter protein, such as DHFR, is restored by reassociation of the first and said second part through binding of the antibody fragment to the (poly)peptide/protein.

It also is an object of the present invention to provide a method, where the first and second parts of the reporter protein are DHFR parts derived from murine mDHFR I and mDHFR II.

In accordance with yet another aspect of the present invention, there are provided methods for the selection and screening of an antibody fragment library for binding to a (poly)peptide/protein in bacterial host cells (e.g., the cytoplasm) by using a Protein Fragment Complementation Assay comprising the steps of: a) introducing into the host cell a first nucleic acid sequence encoding a first fusion protein, where the first fusion protein comprises a first reporter protein part and an antibody fragment, (b) introducing into the host cell a second nucleic acid sequence encoding a second fusion protein, where the second fusion protein comprises a second reporter protein part and an antigen (c) expressing both fusion proteins in the bacterial host cells such that the first and said second fusion proteins can contact each other, wherein reporter protein activity is restored, and (d) screening or selecting for bacterial host cells. In one embodiment, the reporter protein is mDHFR. The reporter protein parts may be MDHFR I and mDHFR II. Either reporter protein part may be linked either to the antibody fragment or to the antigen. The nucleic acid sequences encoding the first and second fusion proteins may be introduced into the host cells in any order, or simultaneously.

It also is an aspect of the present invention to provide a first linker connecting the first part of the reporter protein to the antibody fragment, where the first linker comprises a hydrophilic peptide sequence. The hydrophilic peptide sequence may be (Gly₄Ser)ₙ, wherein n is between 0 and 6. In one embodiment n is 4.

It also is an aspect of the present invention to provide a second linker connecting the second part of the reporter molecule to the (poly)peptide/protein or antigen, where the second linker comprises a hydrophilic peptide sequence. The hydrophilic peptide sequence may be (Gly₄Ser)ₙ, where n is between 0 and 6. In one embodiment n is 2.

In accordance with these and other objects, there are provided a first linker comprising the sequence (Gly₄Ser)₄ and a second linker comprising the sequence (Gly₄Ser)₂.

In accordance with another aspect of the present invention, there are provided antibody fragment libraries comprising, e.g., scFv fragments.

It is another object of the present invention to provide a nucleic acid sequence encoding a fusion protein comprising (i) a first or second part of a reporter protein, (ii) optionally a linker, and (iii) an antibody fragment. The first or second part of the reporter protein linker is fused to the antibody fragment via the linker, if present. The first or second part of the reporter protein may be part of a DHFR molecule, for example mDHFR I or mDHFR II, respectively.

It is yet another object of the present invention to provide a nucleic acid sequence encoding a fusion protein comprising (i) a first or second part of a reporter protein, (ii) optionally a linker, and (iii) a polypeptide capable of binding to an antibody fragment. The first or second part of the reporter protein linker is fused to the polypeptide capable of binding to the antibody fragment fragment via the linker, if present. The first or second part of the reporter protein may be part of a DHFR molecule, for example mDHFR I or mDHFR II, respectively.

It is still a further object of the invention to provide fusion proteins encoded by recombinant DNA molecules of the invention.

It is another object of the present invention to provide a nucleic acid sequence encoding a fusion protein comprising a linker, where the linker comprises the sequence (Gly4Ser)ₙ, where n is between 0 and 6. In one embodiment, n is 4. Where n is 0, there is no linker.

It is yet another object of the present invention to provide a vector comprising the nucleic acid sequences described above, or host cells comprising one or more of the nucleic acid sequences described above, or host cells comprising a vector of the present invention, or kits containing any of the foregoing along with instructions for use.

Further objects, features, aspects, uses and advantages of the present invention will become apparent from the detailed description of preferred embodiments that follows.

### BRIEF DESCRIPTION OF THE FIGURES

FIG. 1 shows a representation of the bacterial enzyme DHFR and the two fragments DHFR I and DHFR II according to Pelletier et al., (1999).

FIG. 2 shows the principle of the Protein Complementation Assay (PCA). The reporter protein, for example the enzyme DHFR from mouse, is dissected into two fragments, named I and II, which are encoded on separate plasmids. Active murine DHFR allows E. coli to grow on minimal medium in the presence of the antibiotic trimethoprim (TMP), which inhibits the bacterial enzyme. Co-transformation of both plasmids and subsequent expression of the fragments by themselves in the bacterial cytoplasm does not lead to functional complementation of DHFR. However, fusion of a linker and an antigen to Fragment I of DHFR and a linker attached to the respective antibody to Fragment II leads to functional complementation of DHFR and therefore to the growth of E. coli on minimal medium in the presence of TMP.

FIG. 3 shows the influence of the linker length between the fusion partners on the efficiency of DHFR complementation. The GCN4/αGCN4 pairs were genetically connected either to fragment I or fragment II of mDHFR, respectively. Plasmids encoding the /αGCN4-scFv fused to fragment II of mDHFR were cotransformed with plasmids encoding the GCN4-zipper domain fused to fragment I of mDHFR (Panel A). Numbers represent the percentage of colonies obtained with one particular pair of plasmids in comparison to the positive control (GCN4 leucine zipper domains fused to either of the two mDHFR fragments). In panel B, the efficiency of cotransformation is shown when the /αGCN4-scFv was fused to fragment I of mDHFR and the GCN-zipper domain fused to fragment II of mDHFR.

FIG. 4 shows a PCR analysis demonstrating that only cognate antigen/antibody pairing gave rise to bacterial growth under the in vivo selection conditions. The antibody and antigen fusions are all cloned in similar vectors (Pelletier et al., 1998; 1999). Thus, if E. coli depends on the presence of both plasmids, PCR with one pair of oligonucleotide primers (annealing outside of the open reading frame) will produce two different bands, one for each plasmid. The presence of the antigen plasmids can be judged simply by their size (indicated at each construct). The PCR products of the antibody fusions all have a similar size and were analyzed by digestion with unique restriction enzymes as indicated in each picture.

### DETAILED DESCRIPTION

The present invention provides methods for the selection or screening of one or more members of an antibody fragment library for binding to a (poly)peptide/protein in bacterial host cells (e.g., the cytoplasm) by using a Protein Fragment Complementation Assay. Briefly, the invention involves the co-expression in a host cell of a first and a second fusion protein, where the first and second fusion protein each comprise two subunits (moieties). The first fusion protein comprises (i) a putative binding protein moiety (e.g., antibody variable domain) fused to (ii) a first subunit of a reporter protein. The second fusion protein comprises (iii) a binding target moiety (e.g., antigen) for the putative binding protein fused to (iv) a second subunit of the reporter protein. The individual subunits of the reporter protein in the fusion proteins are inactive, but binding of the binding protein moiety to the binding target moiety reconstitutes an active reporter activity and provides a detectable signal, demonstrating that the putative binding protein has bound to the binding target.

In one embodiment, the methods use the DHFR Protein as the reporter protein in the Protein Fragment Complementation Assay. A Protein Fragment Complementation Assay has been described, for example, by WO 00/07038.

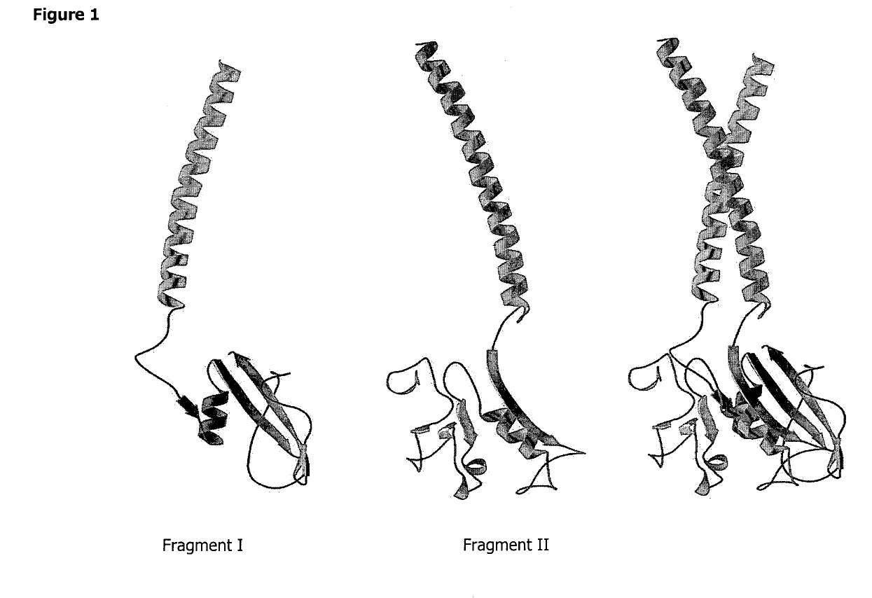

In addition, the invention contemplates the use of a cytoplasmic β-lactamase molecule as a cleavable reporter molecule. For example, Galarneau et al., Nature Biotechnol. 20:619-622 (June, 2002), report on the use of the use of cytoplasmic β-lactamase reporter molecule fragments (based on TEM-β-lactamase) in a PCA, to select for protein-protein interactions. According to FIG. 1 in Galarneau et al., β-lactamase was cleaved into two fragments, designated BLF[1] (amino acids 26-196) and BLF[2] (amino acids 198-290). Galameau et al. dissected β-lactamase between Gly196 and Leu198 because this site is located on a surface opposite to the active site, produces fragments of approximately the same length, contains no periodic secondary structure and is topologically feasible for the protein to fold. Galarneau et al. also created a mutant of the first fragment, BLF[1]MT (M182T), which is known to disrupt an inactive molten-globule intermediate of β-lactamase, reasoning that this mutant could be both more active and metabolically more stable.

Reporter molecules for use in the present inventions are not limited to DHFR, however. As mentioned in WO 00/07038, a particular strategy for designing a protein complementation assay is based on using the following characteristics: A protein or enzyme 1) that is relatively small and monomeric, 2) for which there is a large literature of structural and functional information, 3) for which simple assays exist for the reconstitution of the protein or activity of the enzyme, both in vivo and in vitro, and 4) for which over-expression in eukaryotic and/or prokaryotic cells has been demonstrated.

If some or all of the foregoing selection criteria are met, the structure of the enzyme can be used to decide the best position in the polypeptide chain to split the gene in two, based on the following criteria: 1) the fragments should result in sub-domains of continuous polypeptide; that is, the resulting fragments will not disrupt the subdomain structure of the protein, 2) the catalytic and cofactor binding sites should all be contained in one fragment, and 3) resulting new N- and C-termini should be on the same face of the protein to avoid the need for long peptide linkers and allow for studies of orientation-dependence of protein binding.

The above mentioned criteria need not all be satisfied for operation of the present invention. Advantageously, the enzyme is relatively small, having a molecular weight, for example, between 10-40 kDa. Although monomeric enzymes are preferred, the use of multimeric enzymes is also within the scope of the present invention. For example, the dimeric protein tyrosinase can be used in the methods of the invention.

Information on the structure of the reporter enzyme is an advantage in designing the PCA, but is not essential. Indeed, the present invention provides an additional method to develop PCAs, based on a combination of exonuclease digestion-generated protein fragments followed by directed protein evolution, as exemplified by the enzyme aminoglycoside kinase.

Although the overexpression in prokaryotic cells is preferred it is not a necessity. The skilled artisan also will appreciate that it is not an absolute requirement that the enzyme catalytic site (of the chosen enzyme) be on the same fragment of the dissociated reporter enzyme.

Reporter proteins as described above typically are enzymes that can be divided into two fragments or subunits. Examples of such enzymes are, among others, Dihydrofolate Reductase (DHFR), Aminoglycoside Kinase (AK), Glutathione-S-Transferase (GST) or Green Fluorescent Protein (GFP) (see, e.g., U.S. Pat. No. 6,270,964). Further enzymes that can be modified or adapted to be used in accordance with the present invention (along with selection agents or conditions) include:

| Cleavable Enzyme | Selection Agent/Condition |
|---|---|
| Adenosine deaminase | Xyl-A or adenosine, alanosine, and 2'- |
| | deoxycoformycin |
| Thymidine kinase | gancyclovir, HAT |
| Mutant hypoxanthine- guanine | HAT + thymidine kinase |
| phosphoribosyl transferase | |
| Thymidylate synthetase | 2 fluorodeoxyuridine |
| Xanthine-guanine phosphoribosyl | mycophenolic acid with limiting |
| transferase | xanthine |
| Glutamine synthetase | |
| Asparagine synthetase | B-aspartyl hydroxamate oralbizin |
| Puromycin N-acetyltransferase | Puromycin |
| Aminoglycoside phosphotransferase | Neomycin, G418, gentamycin |
| Hygromycin B | hygromycin B |
| phosphotransferase | |
| L-histidinol:NAD + oxidoreductase | histidinol |
| Bleomycin binding protein | bleomycin/zeocin |
| Cytosine methyl-transferase | 5-Azacytidine (5-aza-CR) and 5-aza-2′- |
| | deoxycytidine |
| 06-alkylguanine alkyltransferase | N-methyl-N-nitrosourea |
| Glycinamide ribonucleotide | dideazatetrahydrofolate, minus purine |
| transformylase | |
| Glycinamide ribonucleotide synthetase | minus purine |
| Phosphoribosyl-aminoimidazole | minus purine |
| synthetase | |
| Formylglycinamide ribotide | L-azaserine, 6-diazo oxo-L-norleucine, |
| amidotransferase | minus purine |
| Phosphoribosyl-aminoimidazole | minus purine |
| carboxamide fonnyltransferase | |
| Phosphoribosyl-aminoimidazole | minus purine |
| Carboxylase | |
| Fatty acid synthase | Cerulenin |
| IMP dehydrogenase | mycophenolic acid |
| Cysteine protease: papain | inhibited by cystatin |
| Cysteine protease: caspase | inhibited by DEVD-aldehyde (can also |
| | by used in a fluorimetric or colorimetric |
| | assay, in vitro) |
| Metalloprotease carboxypeptidase | inhibited by methyl-ethyl succinic acid |
| Serine protease: proteinase K | inhibited by serpins |
| Aspartic protease: pepsin | inhibited by pepstatin A (can also be |
| | used in a fluorimetricassay, in vitro) |
| Lysozyme | inhibited by N-acetylglucosamine |
| | trisaccharide |
| RNAse | inhibited by RNAse inhibitor |
| DNAse | inhibited by actin |
| Phospholipase A2 | many inhibitors, e.g.: bromophenacyl |
| | bromide, hexadecyl-trifluoroethyl- |
| | glycero-phosphomethanol, |
| | bromoenollactone |
| Phospholipase C | many inhibitors, e.g.: |
| | neomycin, chelerythrine, U73122 |
| DT-Diaphorase (NAD(P)H-[quinone | NADPH-diaphorase stain, inhibited by |
| acceptor] oxidoreductase) | dicumarol, Cibacron blue and |
| | phenidione |
| (NAD(P)H-[quinone acceptor] | NRH-diaphorase stain, inhibited by |
| oxidoreductase)-2 | pentahydroxyflavone |
| Thermophilic diaphorase (Bacillus | NADH-diaphorase stain |
| stearothermopizilus) | |
| enicol acetyltransferase | Fluorimetric: Bodipychioramphenicol |
| Unease | Fluorometric |
| SEAP (secreted form of human | chemiluminescent substrate |
| placental alkaline phosphatase) | |
| B-Glucuronidasc | Histochemical, fluorometric or |
| | spectrophotometric assays using |
| | various substrates such as X-GLUC. |

The fragmentation of the reporter proteins causes the loss of the reporter function, but during co-expression of the complementary fusion proteins that contain the reporter protein fragments in host cells, the specific interaction of the two fragments restores reporter protein enzymatic activity. In one embodiment, the reporter protein has enzymatic activity that provides for selection of only those host cells containing reporter protein activity. Successful reassembly of the reporter protein fragments permits growth on a minimal medium, and thus minimizes non-specific interactions and prevents a background of false positives.

Surprisingly, it has been found that parental antibody library members (i.e., antibodies present in a library of antibodies, the members of which have not been optimized for cytoplasmic expression and which still contain cysteine residues) are able to correctly fold in the cytoplasm to the extent necessary and sufficient to restore the function of reporter protein by recognition of the corresponding antigens. This result is particularly surprising because, prior to the present invention, it was not expected that antibody fragments generally could functionally be expressed in the cytoplasm. The surprising discovery of the present invention means that screening of libraries of antibodies to identify antibodies that bind essentially any target antigen by using the PCA has become possible.

Moreover, it has been found that the results obtained with a non-modified antibody library can be improved still further by starting with (or selecting for) an antibody fragment library based on sequences that exhibit or have been optimized to exhibit improved stability and folding efficiency in general. Examples of antibody sequences suitable for such an approach are the consensus-based HuCAL sequences (Knappik et al., 2000). Although such sequences described by Knappik et al. show improved folding, they retain conserved disulfide linkages that generally would not be expected to form correctly in the reducing environment of the cytoplasm of a prokaryotic cell.

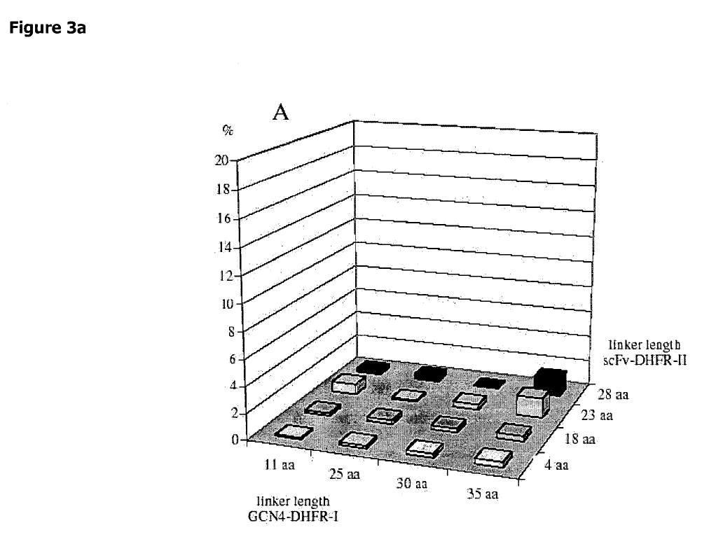

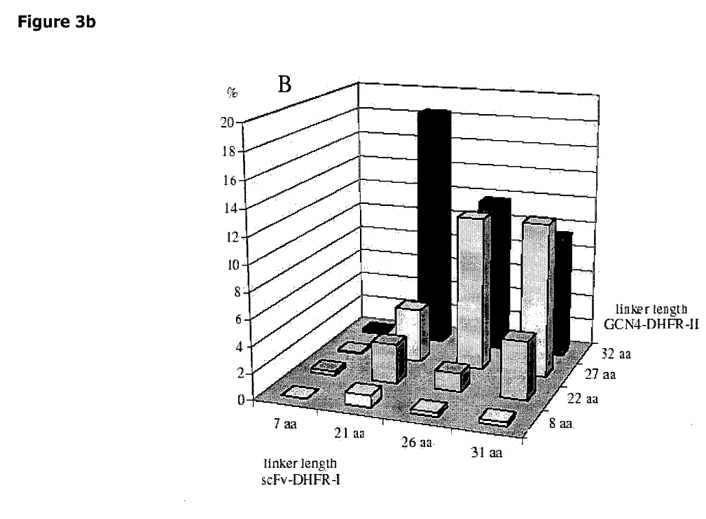

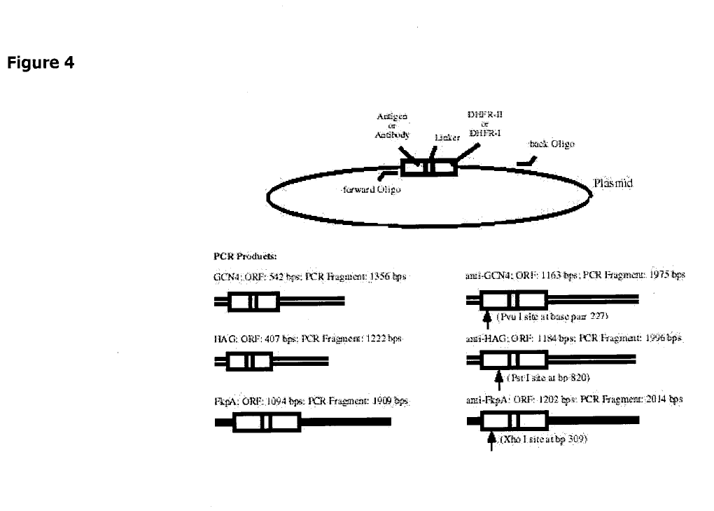

It is additionally surprising that a PCA can be used to screen for antigen-antibody interactions (including the screening of parental antibody library members) because the nature of antibody-antigen interactions (e.g. protein size, tertiary and quarternary structure) is quite distinct from the types of protein-protein interactions heretofore investigated and reported. FIG. 3 illustrates how the linker length between the antibody fragment moiety and reporter molecule fragment can play an important role in reconstitution of reporter molecule activity.

In the context of the present invention, it will be understood that an “antibody” is a protein containing a portion of the variable domains of an immunoglobulin, which portion is sufficient to retain the ability to bind antigen. Suitable antibody fragments for use in the present invention include Fv (Skerra & Plückthun, 1988), scFv (Bird et al., 1988; Huston et al., 1988), disulfide-linked Fv (Glockshuber et al., 1992; Brinkmann et al., 1993), Fab, (Fab′)₂ fragments or other fragments well-known to the practitioner skilled in the art that comprise the variable domain of an antibody or antibody fragment. In a preferred embodiment, the antibody is a Fab fragment, and in a particularly embodiment the antibody is in the scFv fragment format. The antibodies or fragments thereof described herein characteristically contain conserved disulfide bonds characterizing the immunoglobulin fold.

The terms “antibody library” or “antibody fragment library” refer to one or more recombinant libraries of antibodies or fragments thereof, of the types that have been previously described (see, e.g. Vaughan et al., 1996; Knappik et al., 2000; WO 97/08320; Söderlind et al., 2000; Pini et al., 1998; Braunagel et al., 1997) and are well known to one of ordinary skill in the art.

The present invention has been exemplified by the screening of antibody fragment libraries. However, the skilled artisan will appreciate that the invention generally is applicable to the screening or selection of libraries of binding proteins, where the binding proteins in their functional form contain disulfide bonds, and where proper formation of the disulfides is required for correct folding of the proteins in functional form. This includes, for example, human growth hormone and other proteins which are well known to the ordinary practitioner in the art of prokaryotic protein expression.

The term “bacterial host cell” refers herein to bacteria, such as E. coli, used in the production of antibody fragments (see e.g., Ge et al., 1995).

The term “(poly)peptide” refers to molecules consisting of one or more chains of multiple, i.e. two or more, amino acids linked via peptide bonds.

The term “protein” refers to (poly)peptides where at least part of the (poly)peptide has, or is able to acquire, a defined three-dimensional arrangement by forming secondary, tertiary and/or quaternary structures within and/or between its (poly)peptide chain(s). Included within this definition are proteins such as naturally occurring or at least partially artificial proteins, as well as fragments or domains of whole proteins, as long as these fragments or domains are able to acquire a three-dimensional arrangement as described above.

In a particular embodiment of the present invention, the method uses bacterial host cells comprising:

(a) a first nucleic acid sequence encoding a first fusion protein comprising:

(i) a first part of a DHFR molecule fused via,

(ii) optionally a first linker, to

(iii) an antibody fragment representing a member of an antibody fragment library, and

(b) a second nucleic acid sequence encoding a second fusion protein comprising:

(iv) a second part of the DHFR molecule fused via

(v) optionally a second linker to

(vi) a (poly)peptide/protein, and

(c) where the first and said second part of the DHFR molecule are able to restore DHFR activity when the antibody fragment binds to the (poly)peptide/protein.

Methods for constructing nucleic acid molecules encoding the fusion proteins according to the present invention, for construction of vectors comprising these nucleic acid molecules, for introduction of the vectors into appropriately chosen host cells, and for causing or allowing the expression of the (poly)peptides/proteins are well-known in the art (see, e.g., Sambrook et al., 1989; Ausubel et al., 1999; Ge et al., 1995).

In the context of the present invention, the first part of the DHFR molecule and the second part of the DHFR molecule refer to fragments of the enzyme dihydrofolate reductase and, in one embodiment, refer to the murine enzyme, mDHFR.

In the context of the present invention it will be understood that when the said first and second parts of the DHFR molecule are said to be able to restore DHFR activity through binding of the antibody fragment to the (poly)peptide/protein, this refers to the binding or association between the first and second fragments of the DHFR molecule, thereby re-establishing the enzymatic activity of the DHFR molecule.

In the context of the present invention, a linker refers to a polypeptide sequence linking the N- or the C-terminal part of the DHFR fragment to an antibody fragment or a (poly)peptide/protein. Such linkers usually are comprised of about 0 to 100 amino acids, preferably 10 to 50 amino acids and more preferably 10 to 30 amino acids. The linker can be a hydrophilic sequence, such as the type of sequence used to link the variable region chains in ScFv molecules. Such sequences are well known in the art and contain, for example, mixtures of glycine and serine molecules in varying but defined ratios. In one embodiment that linker contains one or more repeating units containing four glycine and one serine residue, e.g. -gly-gly-gly-gly-ser-.

In the context of the present invention, a “first linker” refers to the chain of amino acids that joins the antibody fragment to its DHFR domain fusion partner, and a “second linker” refers to the chain of amino acids that joins the target (poly)peptide/protein and its DHFR domain fusion partner.

In the context of the present invention, binding of the antibody to the target (poly)peptide/protein refers to the interaction between a (poly)peptide/protein, e.g. an antigen and its cognate antibody (and vice versa).

In one embodiment, the first part of the DHFR molecule is mDHFR I, and the second part is mDHFR II. “mDHFR I” and “mDHFR II” are described in U.S. Pat. No. 6,270,964.

In a further embodiment of the present invention, the method comprises the steps of:

(a) introducing into a population of host cells a population of first nucleic acid sequences, each encoding a first fusion protein,

(b) introducing into the host cells a second nucleic acid sequence encoding a second fusion protein, and

(c) expressing the fusion proteins in the bacterial host cells where the first and second fusion proteins may contact each other, and

(d) screening or selecting for bacterial host cells wherein DHFR activity is restored.

The population of host cells may contain, on average, no more than one type of first nucleic acid sequence. In the context of the present invention, the phrases “expressing the fusion proteins” and “allowing the expression” refer to cultivating host cells under conditions such that proteins are expressed.

The phrase “allowing said first and said second fusion proteins to contact each other” refers to allowing the interaction between a (poly)peptide/protein, e.g. an antigen, and its cognate antibody fragment.

In a particular embodiment of the present invention, the first linker comprises the sequence (Gly₄Ser)ₙ, where n is between 0 and 6 or about 6. In a particular embodiment, n=4. In a further embodiment, the second linker comprises the sequence (Gly₄Ser)ₙ, where n is between 0 and 6. In a particular embodiment, n=2. In yet another embodiment of the present invention, the first linker comprises the sequence (Gly₄Ser)₄ and the second linker comprises the sequence (Gly₄Ser)₂.

In a still further embodiment, the present invention relates to an antibody fragment library comprising scFv fragments. In this context, the term “scFv” refers to a fragment, in which the VL and VH chains are joined, in either a VL-VH or VH-VL orientation, by a peptide linker.

In a further aspect, the invention provides nucleic acid sequences encoding a fusion protein comprising:

(i) a first part of a DHFR molecule,

(ii) optionally a linker, and

(iii) an antibody fragment.

In a particular embodiment, the first part of a DHFR molecule is mDHFR I. In another particular embodiment the linker comprises the sequence (Gly₄Ser)ₙ, wherein n is between 0 and 6. In one embodiment n=4.

The present invention is well suited for use in recombinant expression methodologies. In this setting, the present invention relates to a vector comprising a nucleic acid sequence as described above. Also, the present invention provides host cells comprising a nucleic acid sequence or a vector as described above. Further, the invention provides kits for practicing the present invention, containing vectors suitable for expressing fusion proteins comprising reporter fragments as described above. The vectors may be engineered with suitable or convenient restriction endonuclease sites to permit cloning of antibodies and antigens in frame with the reporter protein fragments via an optional linker sequence. The kits may also contain suitable host cells for expressing the fusion proteins.

The invention further is illustrated by the following example, which is intended to illustrate and, hence, not limit the invention.

### EXAMPLE

In the following example, all molecular biology experiments are performed according to standard protocols (see, for example, Ausubel et al., 1999).

Construction of mDHFR Domain Fusion Partner with Varying Linker Length

To test the suitability of scFv antibodies in the PCA system, a positive control with this system was first established using the anti-GCN4 scFv antibody (Wörn et al., 2000) fused to one DHFR domain and the GCN4-leucine zipper fused to the other DHFR domain. This anti-GCN4 scFv is directed against a random coil variant of the dimerization domain of the GCN4 leucine-zipper motif (Hanes et al., 1998) and was modified by grafting its CDR (complementarity determining region) loops to a stability-engineered framework (Wörn et al., 2000). The mDHFR-I and II constructs were prepared as described by Pelletier et al., (1998). Contransformation of plasmids was performed by electroporation of 10 ng of each plasmid into BL21/pREP4 cells (Qiagen). Transformed cells were plated on M9 minimal agar plates in the presence of 2 μg/ml trimethoprim (TMP), 1 mM IPTG, 50 μg/ml kanamycin and 100 μg/ml ampicillin. Numbers of colonies were counted after 4 days of growth at 25° C. The same experiment was performed in the absence of IPTG, to show that TMP-resistance only occurred via expression of the fusion partners and no colonies were observed (data not shown).

The fusions of the antibody and the antigen were optimized by variation of the linker length between the antigen or antibody and its DHFR domain fusion partner and by swapping the DHFR domains. For this purpose, four different linker lengths between each DHFR fragment and its fusion partner were tested in order to find an optimum between possible geometrical restrictions and an unnecessary reduction in effective local concentration through the use of excessively long linkers. Linkers of the sequence (Gly₄Ser)ₙ, with n=0, 2, 3 or 4 were used. The use of the wild-type GCN4 zipper-domain and a mutant (named 7P14P) was also tested, which was the original antigen and carries two additional prolines to disrupt the helical structure of the zipper and therefore prevents its coiled-coil mediated homodimerization (Leder et al., 1995).

Cotransformation of the Different mDHFR Mutants

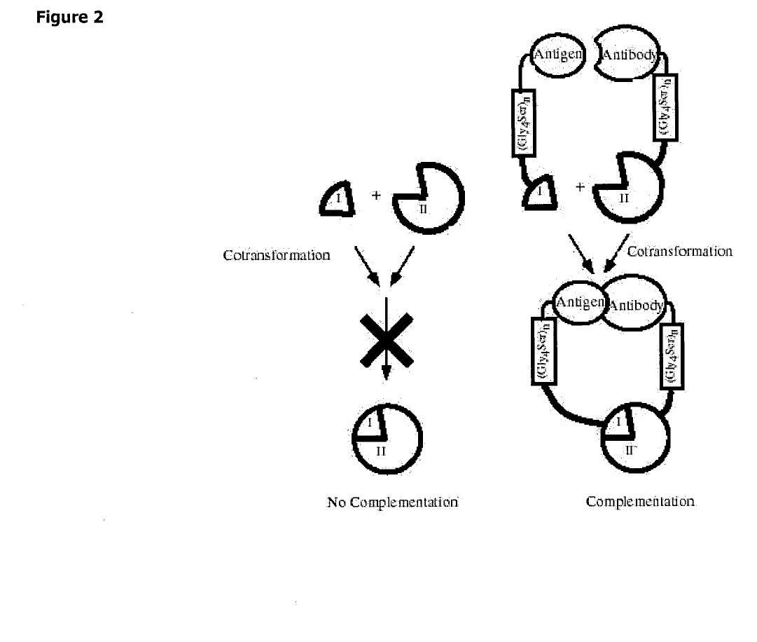

Each GCN4-mDHFR-I fusion construct bearing one of the different linkers was cotransformed in E. coli cells in a pairwise manner with constructs bearing the different anti-GCN4-mDHFR-II fusions (see FIGS. 2A and 2B). The transformed bacteria were plated on M9 minimal agar in the presence of the antibiotic trimethoprim, which inhibits the bacterial DHFR, therefore allowing cells to grow only as a result of functional complementation of the two mDHFR fragments. The constructs of GCN4-mDHFR-II with all different linkers were similarly cotransformed with all anti-GCN4-mDHFR-I constructs. Thus, all linker lengths were tested versus each other. The efficiency of cotransformation with each set was quite asymmetric: the scFv-mDHFR-II fusions, together with the GCN4-mDHFR-I fusions (FIG. 2A), gave rise to far fewer colonies than the opposite pairing (FIG. 2B). The exact reason for this is not known, but one could speculate that one type of constructs shows a stronger aggregation tendency which decreases complementation due to lower availability of one of the active components. Nevertheless, Western blot analysis of all constructs showed no significant differences in their expression patterns. In all cases, the majority of the protein was found to be in the insoluble lysate fraction, with only a minor fraction soluble (data not shown).

For further experiments, the linker pairing and fusion partners that had resulted in the highest number of clones were used. This was the anti-GCN4-mDHFR-I construct with the (Gly₄Ser)₂ linker and the GCN4-mDHFR-II fusion with a (Gly₄Ser)₄ linker (see FIG. 2 B). In this case, over 18% of the colony numbers of the homo-dimerizing zipper-domains of GCN4 were obtained. The same optimization of linker length and orientation of antigen and antibody on DHFR-I and DHFR-II was independently performed with the 7P14P mutant of the GCN4 leucine-zipper, and a similar linker optimum was obtained as with the wild-type GCN4 (data not shown), but the absolute number of colonies increased even to up to 75% of the positive control. This result shows that under these conditions antibodies can be selected just as effectively as interacting helical peptides.

Functionality of the Antigen/Antibody Pairs

In the next step, a test whether other antigen/antibody pairs would also be functional in this assay and whether the antibodies are specific was performed. Therefore, several candidate scFv antibodies were chosen, directed against peptides and whole proteins. The anti-HAG antibody 17/9 used here (Schulze-Gahmen et al., 1993; Krebber et al., 1995), recognizes a 6-amino acid peptide from the haemagglutinin protein of influenza virus and was evolved, using ribosome display, for enhanced stability in the presence of the disulfide reductant DTT (Jermutus et al., 2000). Antibodies that recognize folded proteins as their antigen were also investigated. Four different scFvs were used that were obtained by phage panning of the HuCAL-library (Knappik et al., 2000) versus the peptidyl-prolyl cis/trans isomerase FkpA from E. coli (Missiakas et al., 1996; Bothmann & Plückthun, 2000; Ramm & Plückthun, 2000). All five antibodies and their two antigens were cloned into the vectors with the optimal linker length as determined in the previous experiment with GCN4 and anti-GCN4.

For the complementation assays each antibody containing plasmid was co-transformed with a plasmid encoding its cognate antigen and plated under selective conditions with expression of the fusions induced. The resulting colony numbers are shown in Table 1. The results show that the GCN4/anti-GCN4 system and also the HAG/anti-HAG system gives rise to a large number of clones (about 10⁸ per μg of DNA) when co-transfonned with the respective cognate partner, and therefore support the reassembly of mDHFR. As a negative control, the same experiment was performed when the cells were plated on M9 agar plates without IPTG, conditions under which no fusion proteins were expressed. Here, the number of clones dropped drastically, by 6 to 7 orders of magnitude. This clearly shows that the expression of the fusion constructs is responsible for the ability to grow on minimal medium in the presence of the antibiotic TMP. In the case of the anti-FkpA, only two of the four scFvs, named 9B3 and 7B2, gave rise to a significant number of colonies. The other clones, 6B1 and 11B4, did not show a number of colonies significantly over the background level. Therefore, these two antibodies are not included in Table 1. Western blot analysis of all four anti-FkpA-mDHFR fusions did not show differences in the expression levels of these constructs.

Specificity of the Antigen/Antibody Recognition

The next question was the specificity of the antigen/antibody recognition under the conditions present in the cytoplasm. Therefore, all plasmids expressing an antibody fusion were mixed in two separate experiments, either with a pool of all plasmids containing the antigen fusions, or with a pool of those plasmids containing all antigens except their respective cognate partner (Table 1). The data show that the pool containing the cognate antigen yielded roughly the same number of colonies as when only the cognate plasmids were used (>10⁷ clones per μg of DNA transformed). In the case of the pool without the cognate pairing, only that amount of clones was observed which corresponds to the background (approx. 10 clones per μg of DNA transformed). These results demonstrate that all antibodies used in this study either interacted specifically with their partner or showed no reactivity at all. This is an important observation, since in the yeast two-hybrid system the large number of false positive results is attributed to non-specific binding of some fusion partners (Uetz et al., 2000).

In order to prove that all clones really did arise due to specific interactions, 10-15 of the clones that appeared in each of the experiments were checked with pooled antigen versus single antibody plasmids for the presence of the correct pair of plasmids. Therefore, these colonies were picked and put into a PCR reaction mix which contained one pair of oligonucleotide primers that would anneal to both plasmids and would therefore yield two different products, one for each plasmid (see FIG. 3). Since the DNA sequences for the different antigens and antibodies all contain different diagnostic restriction sites, the presence of the cognate antigen and antibody DNA could be determined by simple digestion of the resulting PCR products (FIG. 3). In all cases investigated, we only observed DNA products from the specific antigen/antibody pairs.

In a final set of experiments, three plasmids encoding the anti-FkpA, anti-GCN4 and anti-HAG scFvs (fused to DHFR-I) and the three respective antigen fusions to DHFR-II were mixed. This mixture of 6 plasmids, all having ampicillin resistance, can give rise to 6 different single transformants and 15 different double transformants, of which 9 are antibody/antigen pairs and only 3 are cognate pairs. Using the PCR diagnostics described above, in 48 colonies grown under selective conditions in minimal medium, only cognate pairs were obtained (Table 2). All 3 cognate pairs were found, each among colonies of a particular size. This experiment thus shows the first library-versus-library demonstration with antibodies and antigens.

One interesting observation was that the growth rate of the E. coli cells was dependent on the different antigen/antibody pairs that it contained. Colonies that appeared after two days of growth at 25° C. harbored exclusively the two FkpA/anti-FkpA pairs. One day later the GCN4/anti-GCN4 pairing could be observed, and only on the fourth day the HAG/anti-HAG pair became visible. While this may reflect the solubility and folding properties of the DHFR fusions, and possibly the antibody affinities, it is conceivable that the antigen FkpA, reported to have some chaperone activity itself (Bothmann & Plückthun, 2000; Ramm & Plückthun, 2000), would also improve the folding yield of scFv-fusions in the cytoplasm of E. coli. These results clearly show that only a specific interaction of antibody and antigen leads to a functional complementation of the mDHFR system.

The remarkable “signal-to-noise” ratio of 7 orders of magnitude (number of specific vs. unspecific colonies) makes this system very suitable for high-throughput antibody generation in functional genomics. Furthermore, no antigen needs to be functionally expressed, purified and immobilized.

### REFERENCES

Arndt, K. M., Pelletier, J. N., Müller, K. M., Alber, T., Michnick, S. W. & Plückthun, A. (2000). A heterodimeric coiled-coil peptide pair selected in vivo from a designed library-versus-library ensemble. J. Mol. Biol. 295, 627-639.

Ausubel, F. M., Brent, R., Kingston, R. E., Moore, D. D., Sedman, J. G., Smith, J. A. & Struhl, K. eds. (1999). Current Protocols in Molecular Biology. New York: John Wiley and Sons.

Bird, R. E., Hardman, K. D., Jacobson, S., Kaufman, B. M., Lee, S. M., Lee, T., Pope, S. H., Riordan, G. S. & Whitlow, M. (1988). Single chain antigen-binding proteins. Science 242, 423-426.

Bothmann, H. & Plückthun, A. (2000). The Periplasmic Escherichia coli Peptidylprolyl cis,trans-Isomerase FkpA. I. Increased functional expression of antibody fragments with and without cis-prolines. J. Biol. Chem. 275, 17100-17105.

Braunagel, M. & Little, D. (1997). Construction of a semisynthetic antibody library using trinucleotide oligos. Nuc. Ac. Res. 22, 4690-4691.

Brinkmann, U., Reiter, Y., Jung, S., Lee, B. & Pastan, I. (1993). A recombinant immunotoxin containing a disulfide-stabilized Fv fragment. Proc. Natl. Acad. Sci. U.S.A. 90, 7538-7542.

Cattaneo, A. & Biocca, S. (1999). The selection of intracellular antibodies. Trends. Biotechnol. 17, 115-121.

Chen, S. Y., Bagley, J. & Marasco, W. A. (1994). Intracellular antibodies as a new class of therapeutic molecules for gene therapy. Hum. Gene Ther. 5, 595-601.

De Jaeger, G., Fiers, E., Eeckhout, D. & Depicker, A. (2000). Analysis of the interaction between single-chain variable fragments and their antigen in a reducing intracellular environment using the two-hybrid system. FEBS Lett. 467, 316-320.

Frisch, C., Kolmar, H., Schmidt, A., Kleemann, G., Reinhardt, A., Pohl, E., Usón, I., Schneider, T. R. & Fritz, H. J. (1996). Contribution of the intramolecular disulfide bridge to the folding stability of REIv, the variable domain of a human immunoglobulin kappa light chain. Fold. Des. 1, 431-440.

Ge, L., Knappik, A., Pack, A., Freund, C. & Plückthun, A. (1995). Expression antibodies in Escherichia coli. Antibody Engineering. A Practical Approach (Ed. C. A. K. Borrebaeck). IRL Press, Oxford, pp 229-266.

Georgiou, G., Stathopoulos, C., Daugherty, P. S., Nayak, A. R., Iverson, B. L. & Curtiss, R., 3rd. (1997). Display of heterologous proteins on the surface of microorganisms: from the screening of combinatorial libraries to live recombinant vaccines. Nat. Biotechnol. 15, 29-34.

Glockshuber, R., Malia, M., Pfitzinger, I. & Plückthun, A. (1992). A comparison of strategies to stabilize immunoglobulin Fv-fragments. Biochemistry 29, 1362-1366.

Glockshuber, R., Schmidt, T. & Plückthun, A. (1992). The disulfide bonds in antibody variable domains: effects on stability, folding in vitro, and functional expression in Escherichia coli. Biochemistry 31, 1270-1279.

Hanes, J., Jermutus, L., Weber-Bornhauser, S., Bosshard, H. R. & Plückthun, A. (1998). Ribosome display efficiently selects and evolves high-affinity antibodies in vitro from immune libraries. Proc. Natl. Acad. Sci. USA 95, 14130-14135.

Huston, J. S., Levinson, D., Mudgett-Hunter, M., Tai, M. S., Novotny, J., Margolies, M. N., Ridge, R. J., Bruccoleri, R. E., Haber, E. & Crea, R. (1988). Protein engineering of antibody binding sites, recovery of specific activity in an anti-digoxin single-chain Fv analogue produced in Escherichia coli. Proc. Natl. Acad. Sci. U.S.A. 85, 5879-5883.

Jermutus, L., Honegger, A., Schwesinger, F., Hanes, J. & Plückthun, A. (2000). Directed in vitro evolution of protein activity and stability. Proc. Natl. Acad. Sci. USA, in press.

Jermutus, L., Honegger, A., Schwesinger, F., Hanes, J. & Plückthun, A. (2001). Tailoring in vitro evolution for protein affinity or stability. Proc. Natl. Acad. Sci. USA, 98, 75-80.

Knappik, A., Ge, L., Honegger, A., Pack, P., Fischer, M., Wellnhofer, G., Hoess, A., Wölle, J., Plückthun, A. & Virnekäs, B. (2000). Fully synthetic human combinatorial antibody libraries (HUCAL) based on modular consensus frameworks and CDRs randomized with trinucleotides. J. Mol. Biol. 296, 57-86.

Krebber, C., Spada, S., Desplancq, D. & Plückthun, A. (1995). Co-selection of cognate antibody-antigen pairs by selectively-infective phages. FEBS Lett. 377, 227-231.

Leder, L., Berger, C., Bornhauser, S., Wendt, H., Ackermann, F., Jelesarov, I. & Bosshard, H. R. (1995). Spectroscopic, calorimetric, and kinetic demonstration of conformational adaptation in peptide-antibody recognition. Biochemistry 34, 16509-16518.

Martineau, P., Jones, P. & Winter, G. (1998). Expression of antibody fragment at high levels in the bacterial cytoplasm. J. Mol. Biol. 280, 117-127.

Missiakas, D., Betton, J. M. & Raina, S. (1996). New components of protein folding in extracytoplasmic compartments of Escherichia coli SurA, FkpA and Skp/OmpH. Mol. Microbiol. 21, 871-884.

Pelletier, J. N., Arndt, K. M., Plückthun, A. & Michnick, S. W. (1999). An in vivo library-versus-library selection of optimized protein-protein interactions. Nat. Biotechnol. 17, 683-690.

Pelletier, J. N., Campbell-Valois, F. X. & Michnick, S. W. (1998). Oligomerization domain-directed reassembly of active dihydrofolate reductase from rationally designed fragments. Proc. Natl. Acad. Sci. USA 95, 12141-12146.

Pini, A., Viti, F., Santucci, A., Camemolla, B., Zardi, L., Neri, P. & Nerid, D. (1998). Design and use of a phage display library—human antibodies with subnanomolar affinity against a marker of angiogenesis eluted from a two-dimensional gel. J. Biol. Chem. 34, 21769-21776.

Proba, K., Ge, L. & Plückthun, A. (1995). Functional antibody single-chain fragments from the cytoplasm of Escherichia coli: influence of thioredoxin reductase (TrxB). Gene 159, 203-207.

Proba, K., Wörn, A., Honegger, A. & Plückthun, A. (1998). Antibody scFv fragments without disulfide bonds made by molecular evolution. J. Mol. Biol. 275, 245-253.

Ramm, K. & Plückthun, A. (2000). The Periplasmic Escherichia coli Peptidylprolyl cis,trans-Isomerase FkpA. II. Isomerase-independent chaperone activity in vitro. J. Biol. Chem. 275, 17106-17113.

Sambrook, J., Fritsch, E. F. & Maniatis, T. (1989). Molecular Cloning: A laboratory manual, Cold Spring Harbor Laboratory Press, Cold Spring Harbor, USA.

Schulze-Gahmen, U., Rini, J. M. & Wilson, I. A. (1993). Detailed analysis of the free and bound conformations of an antibody. X- ray structures of Fab 17/9 and three different Fab-peptide complexes. J. Mol. Biol. 234, 1098-118.

Skerra, A. & Plückthun, A. (1988). Assembly of a functional immunoglobulin Fv fragment in Escherichia coli. Science 240, 1038-1041.

Smith, G. P. (1985). Filamentous fusion phage: novel expression vectors that display cloned antigens on the virion surface. Science 228, 1315-1317.Trill, J. J., Shatzman, A. R. & Ganguly, S. (1995). Production of monoclonal antibodies in COS and CHO cells. Curr. Opin. Biotechnol. 6, 553-560.

Soderlind, E., Strandberg, L., Jirholt, P., Kobayashi, N., Alexeiva, V., Aberg, A. M., Nilsson, A., Jansson, B., Ohlin, M., Wingren, C., Danielsson, L., Carlsson, R. & Borrebaeck, C. A. (2000). Recombining germline-derived CDR sequences for creating diverse single-framework antibody libraries. Nat. Biotechnol. 8, 852-856.

Uetz, P., Giot, L., Cagney, G., Mansfield, T. A., Judson, R. S., Knight, J. R., Lockshon, D., Narayan, V., Srinivasan, M., Pochart, P., Qureshi-Emili, A., Li, Y., Godwin, B., Conover, D., Kalbfleisch, T., Vijayadamodar, G., Yang, M., Johnston, M., Fields, S. & Rothberg, J. M. (2000). A comprehensive analysis of protein-protein interactions in Saccharoinyces cerevisiae. Nature 403, 623-627.

Vaughan, T. J., Williams, A. J., Pritchard, K., Osborn, J. K., Pope, A. R., Eamshaw, J. C., McCafferty, J., Hodits, R. A., Wilton, J. & Johnson, K. S. (1996). Human antibodies with sub-nanomolar affinities isolated from alarge non-immunized phage display library. Nature Biotechnology 14, 309-314.

Visintin, M., Tse, E., Axelson, H., Rabbitts, T. H. & Cattaneo, A. (1999). Selection of antibodies for intracellular function using a two-hybrid in vivo system. Proc. Natl. Acad. Sci. USA 96, 11723-11728.

Winter, G., Griffiths, A. D., Hawkins, R. E. & Hoogenboom, H. R. (1994). Making antibodies by phage display technology. Annu. Rev. Inimunol. 12, 433-455.

Wirtz, P. & Steipe, B. (1999). Intrabody construction and expression III: Engineering hyperstable V_{H} domains. Protein Science 8: 2245-2250. Cambridge University Press.

Wörn, A., Auf der Maur, A., Escher, D., Honegger, A., Barberis, A. & Plückthun, A. (2000). Correlation between in vitro stability and in vivo performance of anti-GCN4 intrabodies as cytoplasmic inhibitors. J. Biol. Chem. 275, 2795-2803.

What is claimed is:

1. A method for the selection or screening of an antibody fragment library for binding to a (poly)peptide/protein in bacterial host cells comprising the step of employing a Protein Fragment Complementation Assay.

2. The method of claim 1, wherein said Protein Fragment Complementation Assay is a DHFR Protein Fragment Complementation Assay.

3. The method of claim 1, wherein each of the host cells used in said assay comprises:
(a) a first nucleic acid sequence encoding a first fusion protein comprising a first part of a reporter molecule fused to an antibody fragment member of said library, optionally via a first linker, and
(b) a second nucleic acid sequence encoding a second fusion protein comprising a second part of said reporter molecule fused to said (poly)peptide/protein optionally via a second linker, and
(c) wherein said first and said second parts of said reporter molecule are able to restore reporter molecule activity by reassociation of said first and said second parts through binding of said antibody fragment to said (poly)peptide/protein.

4. The method of claim 3, wherein said reporter molecule is DHFR.

5. The method according to claim 4, wherein said first part of said DHFR molecule is mDHFR I, and wherein said second part is mDHFR II.

6. A method for the selection or screening of an antibody fragment library for binding to a (poly)peptide/protein in bacterial host cells, comprising the steps of:
(a) expressing in said host cell
(i) a first nucleic acid sequence encoding a first fusion protein comprising a first part of a reporter molecule fused to an antibody fragment member of the library, optionally via a first linker, and
(ii) a second nucleic acid sequence encoding a second fusion protein comprising a second part of a reporter molecule fused to the (poly)peptide/protein, optionally via a second linker;
(b) allowing said first and said second fusion proteins to contact each other, and
(c) screening or selecting for bacterial host cells containing restored reporter molecule activity.

7. The method according to claim 6, wherein said first part of said reporter molecule is fused to said antibody fragment member via a first linker.

8. The method according to claim 7, wherein said second part of said reporter molecule is fused to said (poly)peptide/protein member via a second linker.

9. The method according to claim 8, wherein said reporter molecule is a DHFR molecule.

10. The method according to claim 3, wherein said first linker comprises the sequence (Gly₄Ser)ₙ, wherein n is between 0 and 6.

11. The method according to claim 10, wherein n=4.

12. The method according to claim 3, wherein said second linker comprises the sequence (Gly₄Ser)ₙ, wherein n is between 0 and 6.

13. The method according to claim 12, wherein n=2.

14. The method according to claim 3, wherein said first linker comprises the sequence (Gly₄Ser)₄ and wherein said second linker comprises the sequence (Gly₄Ser)₂.

15. The method according to claim 2, wherein said antibody fragment library comprises scFv fragments.

16. A nucleic acid sequence encoding a fusion protein comprising:
(i) a first part of a reporter molecule,
(ii) optionally a linker, and
(iii) an antibody or a functional antibody fragment.

17. The nucleic acid sequence according to claim 16, wherein said fusion protein comprises a linker.

18. A nucleic acid molecule according to claim 17, wherein said reporter molecule is a cleavable enzyme.

19. A nucleic acid molecule according to claim 18, wherein said reporter molecule is a DHFR molecule.

20. A nucleic acid according to claim 19, wherein said first part of a DHFR molecule is mDHFR I.

21. A nucleic acid according to claim 19, wherein said linker comprises the sequence (Gly₄Ser)ₙ, wherein n is between 0 and 6.

22. A nucleic acid according to claim 21, wherein n=4.

23. A vector comprising the nucleic acid sequence according to claim 16.

24. A bacterial host cell comprising a nucleic acid sequence according to claim 16.

25. A bacterial host cell comprising a vector according to claim 23.

26. A fusion polypeptide comprising
a. a reporter molecule fragment moiety,
b. an antibody or functional antibody fragment moiety, and
c. optionally a linker positioned between said moieties.

27. A fusion protein according to claim 26, comprising a linker positioned between said reporter molecule fragment moiety and said antibody or said functional antibody fragment moiety.

28. A composition of at least two polypeptide chains comprising
a. a first fusion polypeptide comprising
i. a first reporter molecule fragment moiety,
ii. an antibody or functional antibody fragment moiety, and
iii. optionally a first linker positioned between said moieties; and
b. a second fusion polypeptide comprising
i. a second reporter molecule fragment moiety,
ii. an antigen moiety, and
iii. optionally a second linker positioned between said moieties,
wherein said first and second fusion polypeptides interact via said antibody moiety and said antigen moiety, and
wherein said first and second reporter molecule fragment moieties associate to form a functional reporter molecule upon association of said antibody moiety and said antigen moiety.

29. A composition according to claim 28, wherein said first fusion polypeptide comprises a first linker positioned between said first reporter molecule fragment moiety and said antibody or functional antibody fragment moiety.

30. A composition according to claim 29, wherein said second fusion polypeptide comprises a second linker positioned between said second reporter molecule fragment moiety and said antigen moiety.

31. A kit comprising
(a) a plurality of nucleic acid sequences capable of encoding a fusion protein, each of said fusion proteins comprising:
(i) a first part of a reporter molecule,
(ii) optionally a linker, and
(iii) an antibody or functional antibody fragment; and
(b) instructions for using said nucleic acid sequences to isolate an antigen of interest.

32. A kit according to claim 31, wherein each of said fusion proteins comprises a linker.

33. A kit according to claim 32, wherein said antibody fragments represent an antibody library.

34. A kit according to claim 32, wherein said reporter molecule is a DHFR molecule.

35. A method for adapting members of a library of nucleic acids for use in a protein complementation assay, comprising the step of:
modifying the members of said library to encode a fragment of a reporter molecule, wherein said members encode an antibody or functional fragment thereof.

36. A method according to claim 35, wherein said reporter molecule is a DHFR molecule.

37. A method according to claim 36, wherein said fragment of DHFR is mDHFR I or mDHFR II.

38. The method according to claim 8, wherein the reporter molecule is a beta-lactamase molecule.

39. A kit according to claim 32, wherein said reporter molecule is a beta-lactamase molecule.

40. A method according to claim 35, wherein said reporter molecule is a β-lactamase molecule.
